(19) United States
(12) Patent Application Publication
(10) Pub. No.: US 2002/0077467 A1
(43) Pub. Date: Jun. 20, 2002

(54) Mammalian calcitonin-like polypeptide-1

(75) Inventors: Paul O. Sheppard, Granite Falls, WA (US); Emma E. Moore, Seattle, WA (US); Fenella Raymond, Seattle, WA (US)

Correspondence Address: Paul G. Lunn, Esq., ZymoGenetics, Inc., 1201 Eastlake Avenue East, Seattle, WA, 98102, US

(73) Assignee: ZymoGenetics, Inc.

(21) Appl. No.: 09/846,573

(22) Filed: May 1, 2001

Related U.S. Application Data
(63) Continuation-in-part of application No. 09/213,634, filed on Dec. 18, 1998, now abandoned.
(60) Provisional application No. 60/069,976, filed on Dec. 18, 1997.

(51) Int. Cl. C07H 21/04; C07K 14/705; C12P 21/02; C12N 5/06

(52) U.S. Cl. 536/23.5; 530/350; 435/320.1; 435/325; 435/69.1

(57) ABSTRACT

Novel mammalian calcitonin-like polypeptides, polynucleotides encoding the polypeptides, and related compositions and methods including antibodies and anti-idiotypic antibodies.

This is a continuation under 35 U.S.C. §120 of U.S. patent application Ser. No. 09/213,634, which claims priority under 35 U.S.C. §119(e) to U.S. Provisional Application No. 60/069,976 filed Dec. 18, 1997.

### BACKGROUND OF THE INVENTION

Proliferation and differentiation of cells of multicellular organisms are controlled by hormones and polypeptide growth factors. These diffusable molecules allow cells to communicate with each other and act in concert to form cells and organs, and to repair and regenerate damaged tissue. Examples of hormones and growth factors include the steroid hormones (e.g. estrogen, testosterone), parathyroid hormone, follicle stimulating hormone, the interleukins, platelet derived growth factor (PDGF), epidermal growth factor (EGF), granulocyte-macrophage colony stimulating factor (GM-CSF), erythropoietin (EPO) and calcitonin.

Hormones and growth factors influence cellular metabolism by binding to proteins. Proteins may be integral membrane proteins that are linked to signaling pathways within the cell, such as second messenger systems. Other classes of proteins are soluble molecules, such as the transcription factors.

Of particular interest are polypeptides like calcitonin and calcitonin gene-related peptide (CGRP) which can be used to treat bone-related disorders or vascular disorders. Even though these peptides are useful, there use is limited by their marginal effectiveness. Thus, there is a need to discover or develop new peptides which may be useful in treating bone and vascular disorders.

### SUMMARY OF THE INVENTION

The present invention addresses this need by providing a novel polypeptide and related compositions and methods. Within one aspect, the present invention provides an isolated polynucleotide encoding a mammalian cytokine termed ‘Calcitonin-like polypeptide-1, or ‘Zcalc1’. A signal sequence extends from amino acid residue 1 through amino acid residue 21, an alanine, of SEQ ID NO:2. This results in a naturally occurring mature sequence extending from amino acid residue 22, a glycine through amino acid residue 83, a serine, of SEQ ID NO:2, also defined by SEQ ID NO:13. Further processing at the carboxyl terminus results in a mature sequence extending from amino acid residue 22, a glycine, extending through and including amino acid residue 70, a threonine, of SEQ ID NO:2, also defined by SEQ ID NO:14. An active human Zcalc1 polypeptide is also comprised of a sequence of 33 amino acids represented by the amino acid sequence comprised of residues extending from amino acid residue 38, a cysteine residue, through amino acid residue 70, a threonine of SEQ ID NO: 2 and also by SEQ ID NO: 5. In a preferred embodiment the threonine at residue 33 of SEQ ID NO:5 is amidated. Within an additional embodiment, the polypeptide further comprises an affinity tag. Within a further embodiment, the polynucleotide is DNA.

Another allele of Zcalc1 has been cloned and is defined by SEQ ID NOs: 10 and 11. A signal sequence extends from amino acid residue 1 through and including amino acid residue 21, alanine of SEQ ID NO:11. Cleavage of the signal sequence results in a mature sequence which extends from amino acid residue 22, a glycine, extending through amino acid residue 248, a leucine of SEQ ID NO:11. An active portion of this allele of Zcalc1 is comprised of amino acid residue 38, cysteine, through amino acid residue 248, a leucine, of SEQ ID NO:11. This polypeptide is also represented by SEQ ID NO:12.

The following conservative substitutions can be made in SEQ ID NO:2 and SEQ ID NO:11 and Zcalc1 activity will remain. A Trp can be inserted into position 31 in place of the Thr (which is amino acid residue 10 for SEQ ID NOs 12, 13, 14 and 15); a Val or a Thr can be inserted at position 32 in place of the Glu residue (which is amino acid residue 11 for SEQ ID NOs 12, 13, 14 and 15); a Phe can be inserted at position 33 in place of the Arg residue (which is amino acid residue 12 for SEQ ID NOs 12, 13, 14 and 15); a Met can be inserted at position 34 in place of the Leu (which is amino acid residue 13 for SEQ ID NOs 12, 13, 14 and 15); a Thr can be inserted at position 36 in place of the Ser (which is amino acid residue 15 for SEQ ID NOs 12, 13, 14 and 15); an Ile can be inserted at position 40 in place of the Val (which is amino acid residue 19 for SEQ ID NOs 12, 13, 14 and 15). Furthermore, an Ile can be inserted in place of the Val at residue three of SEQ ID NO:5 and Zcalc1 activity will remain. Also claimed are the polynucleotides which encode the above-described variants.

In SEQ ID NO: 11 the following additional changes can be made and Zcalc1 activity will remain. A Glu can be inserted at position 85 in place of the Arg; a Phe can be inserted at position 86 in place of the Leu; an Ile or Ala can be inserted at position 87 in place of the Glu; an Asp can be inserted at position 88 in place of the Glu; a Val or an Ile can be inserted at position 89 in place of the Ala; a Thr can be inserted at position 90 in place of the Leu; a Thr can be inserted at position 92 in place of the Asn; an Ile can be inserted at position 93 in place of the Leu; a Lys or an Asn can be inserted at position 95 in place of the Glu; a Gly can be inserted at position 96 in place of the Arg and a Leu or a Phe can be inserted at position 97 in place of the Ile.

The amino acid residues which are considered important for function in SEQ ID NO:2 and 11 are the Asp at position 30 the Pro at position 35 the Lys at position 37 the Cys at position 38, the Glu at position 39, the Cys at position 41; the Glu at position 47, the Glu at position 91, the Cys at position 94, the Gly at position 108, the Lys at position 129, and the Gly at position 130.

For the embodiment of SEQ ID NO:5 important residues are the Cys at position 1, the Glu at position 2, the Cys at position 4, and the Glu at position 11.

Within a second aspect of the invention there is provided an expression vector comprising (a) a transcription promoter; (b) a DNA segment encoding Zcalc1 polypeptide, and (c) a transcription terminator, wherein the promoter, DNA segment, and terminator are operably linked.

Within a third aspect of the invention there is provided a cultured eukaryotic cell into which has been introduced an expression vector as disclosed above, wherein said cell expresses a protein polypeptide encoded by the DNA segment.

Within a further aspect of the invention there is provided a chimeric polypeptide consisting essentially of a first portion and a second portion joined by a peptide bond. The first portion of the chimeric polypeptide consists essentially of (a) a Zcalc1 polypeptide as shown in SEQ ID NO: 2 (b) allelic variants of SEQ ID NO:5 or SEQ ID NO:12; and (c) protein polypeptides that are at least 90% identical to (a) or (b). The second portion of the chimeric polypeptide consists essentially of another polypeptide such as an affinity tag. Within one embodiment the affinity tag is an immunoglobulin F_{C} polypeptide. The invention also provides expression vectors encoding the chimeric polypeptides and host cells transfected to produce the chimeric polypeptides.

An additional embodiment of the present invention relates to a peptide or polypeptide which has the amino acid sequence of an epitope-bearing portion of a Zcalc1 polypeptide having an amino acid sequence described above. Peptides or polypeptides having the amino acid sequence of an epitope-bearing portion of a Zcalc1 polypeptide of the present invention include portions of such polypeptides with at least nine, preferably at least 15 and more preferably at least 30 amino acids, although epitope-bearing polypeptides of any length up to and including the entire amino acid sequence of a polypeptide of the present invention described above are also included in the present invention. Also claimed are any of these polypeptides that are fused to another polypeptide or carrier molecule. Also claimed are any of these polypeptides that are fused to another polypeptide or carrier molecule. Antibodies produced from these epitope-bearing portions of Zcalc1 can be used in purifying Zcalc1 from cell culture medium.

Within an additional aspect of the invention there is provided an antibody that specifically binds to a Zcalc1 polypeptide as disclosed above, and also an anti-idiotypic antibody which neutralizes the antibody to a Zcalc1 polypeptide.

These and other aspects of the invention will become evident upon reference to the following detailed description and the attached drawing.

### DETAILED DESCRIPTION OF THE INVENTION

The teachings of all of the references cited herein are incorporated herein in their entirety by reference.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems.

“Operably linked”, when referring to DNA segments, indicates that the segments are arranged so that they function in concert for their intended purposes, e.g. transcription initiates in the promoter and proceeds through the coding segment to the terminator.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “soluble protein” is a protein polypeptide that is not bound to a cell membrane.

Within preferred embodiments of the invention the isolated polynucleotides will hybridize to similar sized regions of SEQ ID NO:1, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is about 0.02 M or less at pH 7 and the temperature is at least about 60° C. As previously noted, the isolated polynucleotides of the present invention include DNA and RNA. Methods for isolating DNA and RNA are well known in the art. Total RNA can be prepared using guanidine HCl extraction followed by isolation by centrifugation in a CsCl gradient [Chirgwin et al., Biochemistry 18:52-94 (1979)]. Poly (A)⁺ RNA is prepared from total RNA using the method of Aviv and Leder, Proc. Natl. Acad. Sci. USA 69:1408-1412 (1972). Complementary DNA (cDNA) is prepared from poly(A)⁺ RNA using known methods. Polynucleotides encoding Zcalc1 polypeptides are then identified and isolated by, for example, hybridization or PCR.

Additionally, the polynucleotides of the present invention can be synthesized using a DNA synthesizer. Currently the method of choice is the phosphoramidite method. If chemically synthesized double stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. See Glick, Bernard R. and Jack J. Pasternak, Molecular Biotechnology, Principles & Applications of Recombinant DNA, (ASM Press, Washington, D.C. 1994), Itakura, K. et al. Synthesis and use of synthetic oligonucleotides. Annu. Rev. Biochem. 53: 323-356 (1984), and Climie, S. et al. Chemical synthesis of the thymidylate synthase gene. Proc. Natl. Acad. Sci. USA 87:633-637 (1990).

Those skilled in the art will recognize that the sequences disclosed in SEQ ID NOS:1, 2, 5, 10, 11 and 12 represent alleles of the human. Allelic variants of these sequences can be cloned by probing CDNA or genomic libraries from different individuals according to standard procedures.

The present invention further provides counterpart proteins and polynucleotides from other species (“species orthologs”). Of particular interest are Zcalc1 polypeptides from other mammalian species, including murine, porcine, ovine, bovine, canine, feline, equine, and other primates. Species orthologs of the human Zcalc1 protein can be cloned using information and compositions provided by the present invention in combination with conventional cloning techniques. For example, a cDNA can be cloned using mRNA obtained from a tissue or cell type that expresses the protein. Suitable sources of mRNA can be identified by probing northern blots with probes designed from the sequences disclosed herein. A library is then prepared from mRNA of a positive tissue or cell line. A protein-encoding cDNA can then be isolated by a variety of methods, such as by probing with a complete or partial human or mouse cDNA or with one or more sets of degenerate probes based on the disclosed sequences. A cDNA can also be cloned using the polymerase chain reaction, or PCR (Mullis, U.S. Pat. No. 4,683,202), using primers designed from the sequences disclosed herein. Within an additional method, the cDNA library can be used to transform or transfect host cells, and expression of the cDNA of interest can be detected with an antibody to the protein. Similar techniques can also be applied to the isolation of genomic clones. As used and claimed the language “an isolated polynucleotide which encodes a polypeptide, said polynucleotide being defined by SEQ ID NO: 2” includes all allelic variants and species orthologs of the polypeptide of SEQ ID NO:2.

The present invention also provides isolated protein polypeptides that are substantially identical to the protein polypeptides of SEQ ID NO: 2 and its species orthologs. By “isolated” is meant a protein or polypeptide that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. The term “substantially identical” is used herein to denote polypeptides having 50%, preferably 60%, more preferably at least 80%, sequence identity to the sequence shown in SEQ ID NO:2,or its species orthologs. Such polypeptides will more preferably be at least 90% identical, and most preferably 95% or more identical to SEQ ID NO:2,or its species orthologs. Percent sequence identity is determined by conventional methods. See, for example, Altschul et al., Bull. Math. Bio. 48: 603-616 (1986) and Henikoff and Henikoff, Proc. Natl. Acad. Sci. USA 89:10915-10919 (1992). Briefly, two amino acid sequences are aligned to optimize the alignment scores using a gap opening penalty of 10, a gap extension penalty of 1, and the “blossom 62” scoring matrix of Henikoff and Henikoff (ibid.) as shown in Table 1 (amino acids are indicated by the standard one-letter codes). The percent identity is then calculated as:

$$\frac{\text{Total number of identical matches}}{[ \begin{matrix}\text{length of the longer sequence plus the} \\ \text{number of gaps introduced into the longer} \\ \text{sequence in order to align the two sequences}\end{matrix} ]} \times 100$$

TABLE 1

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Substantially identical proteins and polypeptides are characterized as having one or more amino acid substitutions, deletions or additions. These changes are preferably of a minor nature, that is conservative amino acid substitutions (see Table 2) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A [Nilsson et al., EMBO J. 4:1075 (1985); Nilsson et al., Methods Enzymol. 198:3, 1991), glutathione S transferase (Smith and Johnson, Gene 67:31, (1988), or other antigenic epitope or binding domain. See, in general Ford et al., Protein Expression and Purification 2: 95-107 (1991). DNAs encoding affinity tags are available from commercial suppliers (e.g., Pharmacia Biotech, Piscataway, N.J.).

TABLE 2
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Essential amino acids in the polypeptides of the present invention can be identified according to procedures known in the art, such as site-directed mutagenesis or alanine-scanning mutagenesis [Cunningham and Wells, Science 244: 1081-1085 (1989); Bass et al., Proc. Natl. Acad. Sci. USA 88:4498-4502 (1991)]. In the latter technique, single alanine mutations are introduced at every residue in the molecule, and the resultant mutant molecules are tested for biological activity (e.g., ligand binding and signal transduction) to identify amino acid residues that are critical to the activity of the molecule. Sites of ligand-protein interaction can also be determined by analysis of crystal structure as determined by such techniques as nuclear magnetic resonance, crystallography or photoaffinity labeling. See, for example, de Vos et al., Science 255:306-312 (1992); Smith et al., J. Mol. Biol. 224:899-904 (1992); Wlodaver et al., FEBS Lett. 309:59-64 (1992). The identities of essential amino acids can also be inferred from analysis of homologies with related proteins.

Multiple amino acid substitutions can be made and tested using known methods of mutagenesis and screening, such as those disclosed by Reidhaar-Olson and Sauer, Science 241:53-57, (1988) or Bowie and Sauer, Proc. Natl. Acad. Sci. USA 86:2152-2156 (1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display, e.g., Lowman et al., Biochem. 30:10832-10837 (1991); Ladner et al., U.S. Pat. No. 5,223,409; Huse, WIPO Publication WO 92/06204) and region-directed mutagenesis, Derbyshire et al., Gene 46:145 (1986); Ner et al., DNA 7:127 (1988).

Mutagenesis methods as disclosed above can be combined with high-throughput screening methods to detect activity of cloned, mutagenized proteins in host cells. Preferred assays in this regard include cell proliferation assays and biosensor-based ligand-binding assays, which are described below. Mutagenized DNA molecules that encode active proteins or portions thereof (e.g., ligand-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can prepare a variety of polypeptides that are substantially identical to SEQ ID NO:2 or to SEQ ID NO:5 or allelic variants thereof and retain the properties of the wild-type protein. As expressed and claimed herein the language, “a polypeptide as defined by SEQ ID NO: 2, SEQ ID NO: 5 or SEQ ID NO:11 or 12” includes all allelic variants and species orthologs of the polypeptide.

The protein polypeptides of the present invention, including full-length proteins, protein fragments (e.g. ligand-binding fragments), and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed. (Cold Spring =Harbor Laboratory Press, Cold Spring Harbor, N.Y., 1989), and Ausubel et al., ibid.

In general, a DNA sequence encoding a Zcalc1 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a Zcalc1 polypeptide into the secretory pathway of a host cell, a secretory signal sequence (also known as a leader sequence, prepro sequence or pre sequence) is provided in the expression vector. The secretory signal sequence may be that of the protein, or may be derived from another secreted protein (e.g., t-PA) or synthesized de novo. The secretory signal sequence is joined to the Zcalc1 DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells are preferred hosts within the present invention. Methods for introducing exogenous DNA into mammalian host cells include calcium phosphate-mediated transfection [Wigler et al., Cell 14:725 (1978); Corsaro and Pearson, Somatic Cell Genetics 7:603 (1981); Graham and Van der Eb, Virology 52:456 (1973)], electroporation [Neumann et al., EMBO J. 1:841-845 (1982)], DEAE-dextran mediated transfection [Ausubel et al., eds., Current Protocols in Molecular Biology, (John Wiley and Sons, Inc., NY, 1987)], and liposome-mediated transfection [Hawley-Nelson et al., Focus 15:73, 1993; Ciccarone et al., Focus 15:80 (1993). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134, which are incorporated herein by reference. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 [ATCC No. CRL 1573; Graham et al., J. Gen. Virol. 36:59-72 (1977)] and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. Transformation of insect cells and production of foreign polypeptides therein is disclosed by Guarino et al., U.S. Pat. No. 5,162,222; Bang et al., U.S. Pat. No. 4,775,624; and WIPO publication WO 94/06463. The use of Agrobacterium rhizogenes as a vector for expressing genes in plant cells has been reviewed by Sinkar et al., J. Biosci. (Bangalore) 11:47-58, 1987.

Fungal cells, including yeast cells, and particularly cells of the genus Saccharomyces, can also be used within the present invention, such as for producing protein fragments or polypeptide fusions. Methods for transforming yeast cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in yeast is the POT1 vector system disclosed by Kawasaki et al. (U.S. Pat. No. 4,931,373), which allows transformed cells to be selected by growth in glucose-containing media.

Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465 (1986) and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Within one aspect of the present invention, a novel protein is produced by a cultured cell, and the cell is used to screen for a receptor or receptors for the protein, including the natural receptor, as well as agonists and antagonists of the natural ligand.

Another embodiment of the present invention provides for a peptide or polypeptide comprising an epitope-bearing portion of a Zcalc1 polypeptide of the invention. The epitope of the this polypeptide portion is an immunogenic or antigenic epitope of a polypeptide of the invention. A region of a protein to which an antibody can bind is defined as an “antigenic epitope”. See for instance, Geysen, H. M. et al., Proc. Natl. Acad Sci. USA 81:3998-4002 (1984).

As to the selection of peptides or polypeptides bearing an antigenic epitope (i.e., that contain a region of a protein molecule to which an antibody can bind), it is well known in the art that relatively short synthetic peptides that mimic part of a protein sequence are routinely capable of eliciting an antiserum that reacts with the partially mimicked protein. See Sutcliffe, J. G. et al. Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer soluble peptides, especially those containing proline residues, usually are effective.

Antigenic epitope-bearing peptides and polypeptides of the invention are therefore useful to raise antibodies, including monoclonal antibodies, that bind specifically to a polypeptide of the invention. Antigenic epitope-bearing peptides and polypeptides of the present invention contain a sequence of at least nine, preferably between 15 to about 30 amino acids contained within the amino acid sequence of a polypeptide of the invention. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that react with the protein.

Preferably, the amino acid sequence of the epitope-bearing peptide is selected to provide substantial solubility in aqueous solvents (i.e., the sequence includes relatively hydrophilic residues and hydrophobic residues are preferably avoided); and sequences containing proline residues are particularly preferred. The present invention also provides polypeptide fragments or peptides comprising an epitope-bearing portion of a Zcalc1 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods [see, for example, Geysen et al., supra. See also U.S. Pat. No. 4,708,781 (1987) further describes how to identify a peptide bearing an immunogenic epitope of a desired protein. Antigenic epitope-bearing peptides and polypeptides of the present invention are useful to raise antibodies that bind with the polypeptides described herein which then can be used to purify the protein in either a native or denatured form or to detect the Zcalc1 polypeptide in a western blot.

Protein Isolation:

Expressed recombinant polypeptides (or chimeric polypeptides) can be purified using fractionation and/or conventional purification methods and media. See, for example, Affinity Chromatography: Principles & Methods (Pharmacia LKB Biotechnology, Uppsala, Sweden, 1988). [Methods in Enzymol., Vol. 182: 529-539 “Guide to Protein Purification”, M. Deutscher, (ed.), (Acad. Press, San Diego, 1990,)]. See also Protein Purification Principles and Practice 3^{rd} Edition, Scopes, Robert K. (Springer-Verlag, New York N.Y., 1994). Alternatively, a fusion of the polypeptide of interest and an affinity tag (e.g., polyhistidine, maltose-binding protein, an immunoglobulin domain) may be constructed to facilitate purification.

### Chemical Synthesis of Polypeptides

Polypeptides, especially polypeptides of the present invention can also be synthesized by exclusive solid phase synthesis, partial solid phase methods, fragment condensation or classical solution synthesis. The polypeptides are preferably prepared by solid phase peptide synthesis, for example as described by Merrifield, J. Am. Chem. Soc. 85:2149 (1963). The synthesis is carried out with amino acids that are protected at the alpha-amino terminus. Trifunctional amino acids with labile side-chains are also protected with suitable groups to prevent undesired chemical reactions from occurring during the assembly of the polypeptides.

The alpha-amino protecting group is selectively removed to allow subsequent reaction to take place at the amino-terminus. The conditions for the removal of the alpha-amino protecting group do not remove the side-chain protecting groups. The alpha-amino protecting groups known to be useful in the art of stepwise polypeptide synthesis are acyl type protecting groups (e.g., formyl, trifluoroacetyl, acetyl), aryl type protecting groups (e.g., biotinyl), aromatic urethane type protecting groups [e.g., benzyloxycarbonyl (Cbz), substituted benzyloxycarbonyl and 9-fluorenylmethyloxy-carbonyl (Fmoc)], aliphatic urethane protecting groups [e.g., t-butyloxycarbonyl (tBoc), isopropyloxycarbonyl, cyclohexloxycarbonyl] and alkyl type protecting groups (e.g., benzyl, triphenylmethyl). The preferred protecting groups are tBoc and Fmoc, thus the peptides are said to be synthesized by tBoc and Fmoc chemistry, respectively.

The side-chain protecting groups selected must remain intact during coupling and not be removed during the deprotection of the amino-terminus protecting group or during coupling conditions. The side-chain protecting groups must also be removable upon the completion of synthesis using reaction conditions that will not alter the finished polypeptide. In tBoc chemistry, the side-chain protecting groups for trifunctional amino acids are mostly benzyl based. In Fmoc chemistry, they are mostly tert-butyl or trityl based. In tBoc chemistry, the preferred side-chain protecting groups are tosyl for arginine, cyclohexyl for aspartic acid, 4-methylbenzyl (and acetamidomethyl) for cysteine, benzyl for glutamic acid, serine and threonine, benzyloxymethyl (and dinitrophenyl) for histidine, 2-Cl-benzyloxycarbonyl for lysine, formyl for tryptophan and 2-bromobenzyl for tyrosine. In Fmoc chemistry, the preferred side-chain protecting groups are 2,2,5,7,8-pentamethylchroman-6-sulfonyl (Pmc) or 2,2,4,6,7-pentamethyldihydrobenzofuran-5-sulfonyl (Pbf) for arginine, trityl for asparagine, cysteine, glutamine and histidine, tert-butyl for aspartic acid, glutamic acid, serine, threonine and tyrosine, tBoc for lysine and tryptophan.

For the synthesis of phosphopeptides, either direct or post-assembly incorporation of the phosphate group is used. In the direct incorporation strategy, the phosphate group on serine, threonine or tyrosine may be protected by methyl, benzyl, or tert-butyl in Fmoc chemistry or by methyl, benzyl or phenyl in tBoc chemistry. Direct incorporation of phosphotyrosine without phosphate protection can also be used in Fmoc chemistry. In the post-assembly incorporation strategy, the unprotected hydroxyl groups of serine, threonine or tyrosine are derivatized on solid phase with di-tert-butyl-, dibenzyl- or dimethyl-N,N′-diisopropylphosphoramidite and then oxidized by tert-butylhydroperoxide.

Solid phase synthesis is usually carried out from the carboxyl-terminus by coupling the alpha-amino protected (side-chain protected) amino acid to a suitable solid support. An ester linkage is formed when the attachment is made to a chloromethyl, chlortrityl or hydroxymethyl resin, and the resulting polypeptide will have a free carboxyl group at the C-terminus. Alternatively, when an amide resin such as benzhydrylamine or p-methylbenzhydrylamine resin (for tBoc chemistry) and Rink amide or PAL resin (for Fmoc chemistry) are used, an amide bond is formed and the resulting polypeptide will have a carboxamide group at the C-terminus. These resins, whether polystyrene- or polyamide-based or polyethyleneglycol-grafted, with or without a handle or linker, with or without the first amino acid attached, are commercially available, and their preparations have been described by Stewart et al., “Solid Phase Peptide Synthesis” (2nd Edition), (Pierce Chemical Co., Rockford, Ill., 1984) and Bayer & Rapp Chem. Pept. Prot. 3:3 (1986); and Atherton et al., Solid Phase Peptide Synthesis: A Practical Approach (IRL Press, Oxford, 1989).

The C-terminal amino acid, protected at the side chain if necessary, and at the alpha-amino group, is attached to a hydroxylmethyl resin using various activating agents including dicyclohexylcarbodiimide (DCC), N,N′-diisopropylcarbodiimide (DIPCDI) and carbonyldiimidazole (CDI). It can be attached to chloromethyl or chlorotrityl resin directly in its cesium tetramethylammonium salt form or in the presence of triethylamine (TEA) or diisopropylethylamine (DIEA). First amino acid attachment to an amide resin is the same as amide bond formation during coupling reactions. In a preferred method activation is accomplished by DCC and DMAP, which activates the protected amino acid to a symmetric anhydride which reacts with the active group on the resin. Following the attachment to the resin support, the alpha-amino protecting group is removed using various reagents depending on the protecting chemistry (e.g., tBoc, Fmoc). Fmoc is generally removed by piperidine. The extent of Fmoc removal can be monitored at 300-320 nm or by a conductivity cell. After removal of the alpha-amino protecting group, the remaining protected amino acids are coupled stepwise in the required order to obtain the desired sequence.

To prevent side reactions from occurring, each coupling reaction can be followed by a capping step. The capping solution (0.5M acetic anhydride, 0.125M DIEA, 0.015M HOBt) caps any unreacted amines. On coupling where efficiencies are 99% or better, capping is not necessary. Aside from preventing side reactions capping may be needed if single or double couplings are not successful. Capping can also help prevent concurrent synthesis of fragmented peptides which are similar in length to the peptide of choice. These fragmented peptides may make the purification difficult.

Various activating agents can be used for the coupling reactions including DCC, DIPCDI, 2-chloro-1,3-dimethylimidium hexafluorophosphate (CIP), benzotriazol-1-yl-oxy-tris-(dimethylamino)-phosphonium hexafluorophosphate (BOP) and its pyrrolidine analog (PyBOP), bromo-tris-pyrrolidino-phosphonium hexafluorophosphate (PyBroP), O-(benzotriazol-1-yl)-1,1,3,3-tetramethyluronium hexafluorophosphate (HBTU) and its tetrafluoroborate analog (TBTU) or its pyrrolidine analog (HBPyU), O-(7-azabenzotriazol-1-yl)-1,1,3,3-tetramethyluronium hexafluorophosphate (HATU) and its tetrafluoroborate analog (TATU) or its pyrrolidine analog (HAPyU). The most common catalytic additives used in oupling reactions include 4-dimethylaminopyridine (DMAP), 3-hydroxy-3,4-dihydro-4-oxo-1,2,3-benzotriazine (HODhbt), N-hydroxybenzotriazole (HOBt) and 1-hydroxy-7-azabenzotriazole (HOAt). Preferably HOBt-HBTU is used with DIEA. Each protected amino acid is used in excess (>2.0 equivalents), and the couplings are usually carried out in N-methylpyrrolidone (NMP) or in DMF, CH₂Cl₂ or mixtures thereof. The extent of completion of the coupling reaction can be monitored at each stage, e.g., by the ninhydrin reaction as described by Kaiser et al., Anal. Biochem. 34:595 (1970). In cases where incomplete coupling is found, the coupling reaction is extended and repeated and may have chaotropic salts added. The coupling reactions can be performed automatically with commercially available instruments such as ABI model 430A, 431A and 433A peptide synthesizers.

After the entire assembly of the desired peptide, the peptide-resin is cleaved with a reagent with proper scavengers. The Fmoc peptides are usually cleaved and deprotected by TFA with scavengers (e.g., H₂O, ethanedithiol, phenol and thioanisole). The tBoc peptides are usually cleaved and deprotected with liquid HF for 1-2 hours at −5 to 0° C., which cleaves the polypeptide from the resin and removes most of the side-chain protecting groups. Scavengers such as anisole, dimethylsulfide and p-thiocresol are usually used with the liquid HF to prevent cations formed during the cleavage from alkylating and acylating the amino acid residues present in the polypeptide. The formyl group of tryptophan and the dinitrophenyl group of histidine need to be removed, respectively by piperidine and thiophenol in DMF prior to the HF cleavage. The acetamidomethyl group of cysteine can be removed by mercury(II)acetate and alternatively by iodine, thallium(III)trifluoroacetate or silver tetrafluoroborate which simultaneously oxidize cysteine to cystine. Other strong acids used for tBoc peptide cleavage and deprotection include trifluoromethanesulfonic acid (TFMSA) and trimethylsilyltrifluoroacetate (TMSOTf).

Uses

Zcalc1 is widely expressed in a number of tissues including fetal brain, placenta, stomach, uterus, prostate, heart, liver, skeletal muscle, kidney, small intestine, colon, adrenal gland and pituitary gland. Zcalc1 has similarities in structure to calcitonin-related peptide (CGRP) and since CGRP is a neuromodulator, Zcalc1 can also be used as a neuromodulator in a variety of peripheral organs, Path. Biol 44(10): 867-874 (1996). CGRP was also shown to promote proliferation of T-cells in murine intestinal smooth muscle cells, therefore, can be used to promote proliferation of T-cells during infection or after chemotherapy or radiological therapy, Hoagaboam, C. M. et al., J. Neuroimmunol. 75: 123-134 (1997). Zcalc1 also has similarities and is about 25% identical to calcitonin. Calcitonin lowers Ca²⁺ and phosphate concentrations in patients with hypercalcemia, the effect of a single dose lasting 6 to 10 hours. This effect results from decreased bone resorption and is greater in patients in whom bone turnover rates are high.

Calcitonin is effective in disorders of increased skeletal remodeling, such as Paget's disease (osteitis deformans), and in some patients with osteoporosis. Paget's disease, osteitis deformans, is a disease of bone marked by repeated episodes of increased mass. There may be bowing of the long bones and deformation of flat bones resulting in possible pain and pathological fractures. The patient is initially treated with 100 units/day of salmon calcitonin, favorable results usually are obtained when dosage is reduced to 50 units three times a week when salmon calcitonin is used. When synthetic human calcitonin is used, the initial subcutaneous dose for Paget's disease is 0.5 mg. As a powerful inhibitor of osteoclastic bone resorption, calcitonin also produces modest increase in bone mass in patients with osteoporosis. Increases are most impressive in patients with high intrinsic rates of bone turnover, approaching 10% to 15% before reaching a plateau. In a similar manner, Zcalc1 can be used to treat these diseases.

Zcalc1 can be prepared in solution and administered subcutaneously at a dose of 0.5 mg/day 2 or 3 days a week in the treatment of Paget's disease, hypercalcemia and osteoporosis. More severe cases may require 1 mg/day (0.5mg twice/day). The serum alkaline phosphatase and urinary hydroxyproline excretion should be determined prior to therapy, during the first 3 months and every 3 to 6 months during chronic therapy. See Goodman & Gilman's The Pharmacological Basis of Therapeutics 9^{th} Ed. (McGraw-Hill, 1995).

Zcalc1 can also be administered to an individual to treat Raynaud's disease in the same way as CGRP can be used. See Bunker, C. B., et al., Lancet 342:80-83 (1993). Raynaud's Phenomenon is a disease characterized by episodic digital ischemia, manifested clinically by the sequential development of digital blanching, cyanosis, and rubor of the fingers or toes following cold exposure and subsequent rewarming. In severe cases Zcalc1 can be administered to the individual in the same manner and dosage range as in the treatment of osteoporosis.

There is also a possibility that Zcalc1 can be used to inhibit the progression of type I diabetes. See Khachatryan A, et al. J. Immunol., 158:1409-1416 (1997) in which nonobese diabetic (NOD) mice were engineered to produce CGRP in pancreatic beta cell by placing a modified CGRP gene under the control of the rat insulin promoter. The production of CGRP by the beta cells prevented insulin-dependent diabetes mellitus in male NOD mice and reduced its incidence by 63% in female NOD mice. Thus, thus Zcalc1 can also be administered to an individual to prevent diabetes mellitus. Zcalc1 should be administered to an individual at the onset of symptoms of type I diabetes to inhibit the CD4 T cell production of the cytokines that have been implicated in the pathology of type I diabetes. Zcalc1 can be prepared in solution and administered subcutaneously at a dose of 0.5 mg/day for 30 days at the onset of symptoms of diabetes mellitus. If inflammation of the pancreas is not reduced, the dose may be increased to lmg/day (0.5 mg twice/day) for an additional 30 days. Administration of Zcalc1 may be needed on a regular but less frequent basis after the symptoms have subsided.

The present invention also provides reagents with significant therapeutic value. The Zcalc1 polypeptide (naturally occurring or recombinant), fragments thereof, antibodies and anti-idiotypic antibodies thereto, along with compounds identified as having binding affinity to the Zcalc1 polypeptide, should be useful in the treatment of conditions associated with abnormal physiology or development, including abnormal proliferation, e.g., cancerous conditions, or degenerative conditions. For example, a disease or disorder associated with abnormal expression or abnormal signaling by a Zcalc1 polypeptide should be a likely target for an agonist or antagonist of the Zcalc1 polypeptide.

Antibodies to the Zcalc1 polypeptide can be purified and then administered to a patient. These reagents can be combined for therapeutic use with additional active or inert ingredients, e.g., in pharmaceutically acceptable carriers or diluents along with physiologically innocuous stabilizers and excipients. These combinations can be sterile filtered and placed into dosage forms as by lyophilization in dosage vials or storage in stabilized aqueous preparations. This invention also contemplates use of antibodies, binding fragments thereof or single-chain antibodies of the antibodies including forms which are not complement binding.

CRGP is also a potent vasodilator and has been associated as a causative agent of menopausal hot flushes, Chen J., et al., Lancet 342:49 (1993) and as a causative agent in flushing associated with neuroendocrine tumors. Thus, antagonists to Zcalc1, e.g., antibodies to Zcalc1, may be administered to an individual to alleviate such flushing. Also, Zcalc1 can be administered as a vasodilator to treat hypertension, i.e. high blood pressure.

There is also data that suggests that CGRP is involved in ultraviolet radiation-induced immunosuppression, Gillardon F., Eur. J. Pharmacol. 293: 395-400 (1995). Thus, antibodies to Zcalc1 can be used to alleviate this immunosuppression; or perhaps Zcalc1 can be used as an immunosuppressive agent to prevent rejection of transplanted organs.

The quantities of reagents necessary for effective therapy will depend upon many different factors, including means of administration, target site, physiological state of the patient, and other medications administered. Thus, treatment dosages should be titrated to optimize safety and efficacy. Typically, dosages used in vitro may provide useful guidance in the amounts useful for in vivo administration of these reagents. Animal testing of effective doses for treatment of particular disorders will provide further predictive indication of human dosage. Methods for administration include oral, intravenous, peritoneal, intramuscular, or transdermal administration. Pharmaceutically acceptable carriers will include water, saline, buffers to name just a few. Dosage ranges would ordinarily be expected from 1 μg to 1000 μg per kilogram of body weight per day. However, the doses by be higher or lower as can be determined by a medical doctor with ordinary skill in the art. For a complete discussion of drug formulations and dosage ranges see Remington's Pharmaceutical Sciences, 18^{th} Ed., (Mack Publishing Co., Easton, Pa., 1995), and Goodman and Gilman's: The Pharmacological Bases of Therapeutics, 9^{th} Ed. (Pergamon Press 1996).

Nucleic Acid-based Therapeutic Treatment

If a mammal has a mutated or lacks a Zcalc1 gene, the Zcalc1 gene can be introduced into the cells of the mammal. In one embodiment, a gene encoding a Zcalc1 polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses , which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector [Kaplitt et al., Molec. Cell. Neurosci., 2:320-330 (1991)], an attenuated adenovirus vector, such as the vector described by Stratford-Perricaudet et al., J. Clin. Invest., 90 :626-630 (1992), and a defective adeno-associated virus vector [Samulski et al., J. Virol., 61:3096-3101 (1987); Samulski et al. J. Virol., 63:3822-3828 (1989)]. Furthermore, the gene can be introduced in a retroviral vector, e.g., as described in Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995 by Dougherty et al.; and Blood, 82:845 (1993).

Alternatively, the vector can be introduced by lipofection in vivo using liposomes. Synthetic cationic lipids can be used to prepare liposomes for in vivo transfection of a gene encoding a marker [Felgner et al., Proc. Natl. Acad. Sci. USA, 84:7413-7417 (1987); see Mackey et al., Proc. Natl. Acad. Sci. USA, 85:8027-8031 (1988)]. The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

It is possible to remove the cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter [see, e.g., Wu et al., J. Biol. Chem., 267:963-967 (1992); Wu et al., J. Biol. Chem., 263:14621-14624 (1988)].

Zcalc1 polypeptides can also be used to prepare antibodies that specifically bind to Zcalc1 polypeptides. These antibodies can then be used to manufacture anti-idiotypic antibodies. As used herein, the term “antibodies” includes polyclonal antibodies, monoclonal antibodies, antigen-binding fragments thereof'such as F(ab′)₂ and Fab fragments, and the like, including genetically engineered antibodies. Antibodies are defined to be specifically binding if they bind to a Zcalc1 polypeptide with a Kₐ of greater than or equal to 10⁷/M. The affinity of a monoclonal antibody can be readily determined by one of ordinary skill in the art (see, for example, Scatchard, ibid.).

Methods for preparing polyclonal and monoclonal antibodies are well known in the art (see for example, Sambrook et al., Molecular Cloning: A Laboratory Manual, Second Edition, (Cold Spring Harbor, N.Y., 1989); and Hurrell, J. G. R., Ed., Monoclonal Hybridoma Antibodies: Techniques and Applications (CRC Press, Inc., Boca Raton, Fla., 1982). As would be evident to one of ordinary skill in the art, polyclonal antibodies can be generated from a variety of warm-blooded animals such as horses, cows, goats, sheep, dogs, chickens, rabbits, mice, and rats. The immunogenicity of a Zcalc1 polypeptide may be increased through the use of an adjuvant such as Freund's complete or incomplete adjuvant. A variety of assays known to those skilled in the art can be utilized to detect antibodies which specifically bind to Zcalc1 polypeptides. Exemplary assays are described in detail in Antibodies: A Laboratory Manual, Harlow and Lane (Eds.), (Cold Spring Harbor Laboratory Press, 1988). Representative examples of such assays include: concurrent immunoelectrophoresis, radio-immunoassays, radio-immunoprecipitations, enzyme-linked immunosorbent assays (ELISA), dot blot assays, inhibition or competition assays, and sandwich assays.

Antibodies are determined to be specifically binding if: 1) they exhibit a threshold level of binding activity, and 2) they do not cross-react with prior art polypeptide molecules. First, antibodies herein specifically bind if they bind to a Zcalc1 polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis.

Second, antibodies are determined to specifically bind if they do not cross-react with polypeptides of the prior art. Antibodies do not significantly cross-react with related polypeptide molecules, for example, if they detect Zcalc1 but not known related polypeptides using a standard Western blot analysis (Ausubel et al., ibid.). Examples of known related polypeptides are orthologs, proteins from the same species that are members of a protein family (e.g. IL-16), Zcalc1 polypeptides, and non-human Zcalc1. Moreover, antibodies may be “screened against” known related polypeptides to isolate a population that specifically binds to the inventive polypeptides. For example, antibodies raised to Zcalc1 are adsorbed to related polypeptides adhered to insoluble matrix; antibodies specific to Zcalc1 will flow through the matrix under the proper buffer conditions. Such screening allows isolation of polyclonal and monoclonal antibodies non-crossreactive to closely related polypeptides, Antibodies: A Laboratory Manual, Harlow and Lane (eds.) (Cold Spring Harbor Laboratory Press, 1988); Current Protocols in Immunology, Cooligan, et al. (eds.), National Institutes of Health (John Wiley and Sons, Inc., 1995). Screening and isolation of specific antibodies is well known in the art. See, Fundamental Immunology, Paul (eds.) (Raven Press, 1993); Getzoff et al., Adv. in Immunol. 43: 1-98 (1988); Monoclonal Antibodies: Principles and Practice, Goding, J. W. (eds.), (Academic Press Ltd., 1996); Benjamin et al., Ann. Rev. Immunol. 2: 67-101 (1984).

A variety of assays known to those skilled in the art can be utilized to detect antibodies which specifically bind to Zcalc1 proteins or peptides. Exemplary assays are described in detail in Antibodies: A Laboratory Manual, Harlow and Lane (Eds.) (Cold Spring Harbor Laboratory Press, 1988). Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant Zcalc1 protein or polypeptide.

Antibodies to Zcalc1 may be used for tagging cells that express the protein, for affinity purification, within diagnostic assays for determining circulating levels of soluble protein polypeptides, and as antagonists to block ligand binding and signal transduction in vitro and in vivo. Anti-idiotypic antibodies can be used to discover a receptor of Zcalc1.

Antibodies to Zcalc1 are may be used for tagging cells that express the protein, for affinity purification, within diagnostic assays for determining circulating levels of soluble protein polypeptides, and as antagonists to block ligand binding and signal transduction in vitro and in vivo.

Radiation hybrid mapping is a somatic cell genetic technique developed for constructing high-resolution, contiguous maps of mammalian chromosomes [Cox et al., Science 250:245-250 (1990)]. Partial or full knowledge of a gene's sequence allows the designing of PCR primers suitable for use with chromosomal radiation hybrid mapping panels. Commercially available radiation hybrid mapping panels which cover the entire human genome, such as the Stanford G3 RH Panel and the GeneBridge 4 RH Panel (Research Genetics, Inc., Huntsville, Ala.), are available. These panels enable rapid, PCR based, chromosomal localizations and ordering of genes, sequence-tagged sites (STSs), and other nonpolymorphic- and polymorphic markers within a region of interest. This includes establishing directly proportional physical distances between newly discovered genes of interest and previously mapped markers. The precise knowledge of a gene's position can be useful in a number of ways including: 1) determining if a sequence is part of an existing contig and obtaining additional surrounding genetic sequences in various forms such as YAC-, BAC- or cDNA clones, 2) providing a possible candidate gene for an inheritable disease which shows linkage to the same chromosomal region, and 3) for cross-referencing model organisms such as mouse which may be beneficial in helping to determine what function a particular gene might have.

The results showed that Zcalc1 maps 3.25 cR_{—}3000 distal from the human chromosome 7 framework marker D7S651 on the WICGR radiation hybrid map. The use of the surrounding markers positions the Zcalc1 gene in the 7q22.1 region on the integrated LDB chromosome 7 map (The Genetic Location Database, University of Southhampton, WWW server: http://cedar.genetics.soton.ac.uk/public_html/).

The present invention also provides reagents which will find use in diagnostic applications. For example, the Zcalc1 gene, a probe comprising Zcalc1 DNA or RNA or a subsequence thereof can be used to determine if the Zcalc1 gene is present on chromosome 7q22.1 or if a mutation has occurred. Detectable chromosomal aberrations at the Zcalc1 gene locus include but are not limited to aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art [Sambrook et al., ibid.; Ausubel, et. al., ibid.; Marian, A. J., Chest, 108: 255-265, (1995)].

### EXAMPLE 1

### Chemical Synthesis of Zcalc1

Synthesis and Purification

Zcalc-1, SEQ ID NO:5 was synthesized by solid phase peptide synthesis using the ABI/PE Peptide Synthesizer model 431A [Applied Biosytems/Perkin Elmer (ABI/PE), Foster City, Calif.] starting with Fmoc-Amide resin. The Fmoc-Amide resin (0.68 mmol/g) was purchased from ABI/PE. The amino acids were purchased from AnaSpec, Inc., San Jose, Calif. in preweighed, 1 mmol cartridges. All the reagents except piperidine were purchased from ABI/PE. The piperidine was purchased from Aldrich, St. Louis Mo. Synthesis procedure was taken from the ABI Model 431A manual. Double coupling cycles were used during the high aggregation portion of the sequence, as predicted by Peptide Companion software (Peptides International, Louisville, Ky.). The double-coupling sites were at amino acid residues 1-3, and amino acid residues 23-24 from the N-terminus. Capping steps were at amino acid residues 11-15 and amino acid residues 23-24 of SEQ ID NO:5.

The peptide was cleaved from the solid phase following the standard TFA cleavage procedure as outlined in the Peptide Cleavage protocol manual published by ABI/PE. Purification of the peptide was by RP-HPLC using a C18, 10·m preparative column. Eluted fractions from the column were collected and analyzed for correct mass and purity by electrospray mass spectrometry. The analysis results indicated that the Zcalc-1 peptide was present and pure in one of the pools. The pool containing the peptide was retained and lyophilized.

Disulfide Bond Formation

To form the disulfide bond between the two cystine residues, the peptide underwent chemical oxidation which breaks the hydrogen bonds and reforms to a disulfide bond. The lyophilized peptide was oxidized overnight in NH₄HCO₃, pH 8.3 and 15% DMSO at a concentration of 1 mg/ml. (This oxidation procedure was developed by J. P. Tam, J. of American Chemistry Society, 1991, 113, 6657-6662). Post oxidation, the peptide was desalted using a C18, 5·m semi-preparative column. The eluted peptide fractions were pooled and analyzed for disulfide content.

Analysis using the MALDI-TOF mass spectrometer indicated that the peptide was present at the correct mass range. Comparison of the oxidized material to the non-oxidized native material indicated that the two peptides were the same.

Disulfide Bond Analysis

An enzyme digestion using endoproteinase Glu-C was performed on both the oxidized and native peptide. The Glu-C enzyme cleaves peptide bonds at the C-terminal of glutamic acid in ammonium carbonate buffer, pH 7.8. Due to the disulfide bond position of the oxidized peptide, the C-E(AA 1-2) fragment would remain attached to the AA fragment 3-8 with an additional mass of 18 (for water). Fragments AA 1-2 and AA 3-8 were not detected indicating proper formation of the disulfide bond between Cys at position 1 and Cys at position 3. The mass spectrometry chromatogram were similar for both the oxidized and the non-oxidized peptide. The polypeptide of SEQ ID NO: 5 was produced in which carboxy-terminus threonine was amidated.

Using the procedure described above, the allelic variant of Zcalc1 of SEQ ID NO: 12 can also be synthesized.

### EXAMPLE 2

A polymerase chain reaction (PCR) was conducted on a number of cDNA libraries which had MARATHON<< (Clontech, Palo Alto, Calif.) linkers ligated onto the DNAs using the primers ZC15,546 (SEQ ID NO:6) and ZC15,547 (SEQ ID NO:7). The PCR reaction conditions were as follows.

The PCR mixture for the reaction contained 40 μl of lOX PCR buffer, 8 μl EXTAG (both from Takara, Madison Wis.), 8 μl of 2.5 mM nucleotide triphosphate mix Takara) and 300 μl of water. The PCR reaction was incubated at 94° C. for 1.5 minutes, and then run for 35 cycles each individual cycle being comprised of 15 seconds at 94° C., 20 seconds at 58° C. and 30 seconds at 72° C. The reaction was ended with an incubation for 10 minutes at 72° C. and a hold at 4° C.

A 360 bp DNA corresponding to SEQ ID NO: 3 was seen in fetal brain, placenta, stomach, uterus and prostate cDNA. A faint band was also seen in brain cDNA.

### EXAMPLE 3

### Northern Blot Analysis

Human multiple tissue blots 1,2,3 (Clontech) were probed to determine the tissue distribution of Zcalc1. The DNA produced in Example 2 was isolated on a 1.0% agarose gel. The DNA was extracted from the gel slab with a QIAquick Gel Extraction Kit (Qiagen). 100 ng of this DNA was labeled with P³² using the REIPRIME<< Labeling System (Amersham) and unincorporated radioactivity was removed with a NucTrap Probe Purification Column (Stratagene). Multiple tissue northerns and a human RNA master blot were prehybridized 3 hours with 10 ml EXPRESSHYB<< Solution (Clontech) containing 1 mg salmon sperm DNA which was boiled 5 minutes and then iced 1 minute and added to 10 ml of ExpressHyb Solution, mixed and added to blots. Hybridization was carried out overnight at 65° C. Initial wash conditions were as follows: 2× SSC, 0.05% SDS RT for 40 minutes with several changes of solution then 0.1× SSC, 0.1% SDS at 50° C. for 40 minutes, 1 solution change. Blots were than exposed to film a −80° C. for 2.5 hours.

A transcript of approximately 0.75 kb was seen in many tissues on the multi-tissue blot, including heart, liver skeletal muscle, kidney, small intestine and colon. The dot blot also had a signal in many tissues including heart, adrenal gland, kidney, liver, small intestine, pituitary gland and colon.

### EXAMPLE 4

### Chromosomal Assignment and Placement of Zcalc1

Zcalc1 was mapped to chromosome 7 using the commercially available “GeneBridge

“4 Radiation Hybrid Panel” (Research Genetics, Inc., Huntsville, Ala.). The GeneBridge 4 Radiation Hybrid Panel contains PCRable DNAs from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of Zcalc1 with the “GeneBridge 4 RH Panel”, 20 ¦l reactions were set up in a PCRable 96-well microtiter plate (Stratagene, La Jolla, Calif.) and used in a “RoboCycler Gradient 96” thermal cycler (Stratagene). Each of the 95 PCR reactions consisted of 2 ¦l 10× KlenTaq PCR reaction buffer (CLONTECH Laboratories, Inc., Palo Alto, Calif.), 1.6 ¦l dNTPs mix (2.5 mM each, PERKIN-ELMER, Foster City, Calif.), 1 ¦l sense primer, (SEQ ID NO: 8) 5′ AGC GGT GAT TGT TTG TAG 3′, 1 ¦l antisense primer, (SEQ ID NO: 9), 5′ TGG GCA AGC GTT CTG TGT 3′, 2 ¦l “RediLoad” (Research Genetics, Inc., Huntsville, Ala.), 0.4 ¦l 50× Advantage KlenTaq Polymerase Mix (Clontech Laboratories, Inc.), 25 ng of DNA from an individual hybrid clone or control and x ¦l ddH₂O for a total volume of 20 ¦l. The reactions were overlaid with an equal amount of mineral oil and sealed. The PCR cycler conditions were as follows: an initial 1 cycle 5 minute denaturation at 95° C., 35 cycles of a 1 minute denaturation at 95° C., 1 minute annealing at 64° C. and 1.5 minute extension at 72° C., followed by a final 1 cycle extension of 7 minutes at 72° C. The reactions were separated by electrophoresis on a 2% agarose gel (Life Technologies, Gaithersburg, Md.).

The results showed that Zcalc1 maps 3.25 cR_{—}3000 distal from the human chromosome 7 framework marker D7S651 on the WICGR radiation hybrid map. The use of surrounding markers positions Zcalc1 in the 7q22.1 region on the integrated LDB chromosome 7 map (The Genetic Location Database, University of Southhampton, WWW server: http://cedar.genetics. soton.ac.uk/public_html/)

We claim:

1. An isolated polynucleotide which encodes a mammalian polypeptide, said polypeptide being comprised of the amino acid sequence of SEQ ID NO: 2, 5, 11, 12, 13, 14 or 15.

2. A polynucleotide of claim 1 wherein the polynucleotide encodes a polypeptide which is at least 90% identical to one of said polypeptides of claim 1.

3. A polynucleotide of claim 1 wherein the polynucleotide encodes a polypeptide of claim 1 having the following amino acid residue variability in SEQ ID NO:2 and 11 the amino acid residue at position 31 (which is amino acid residue 10 for SEQ ID NOs 12, 13, 14 and 15) is Trp or Thr; the amino acid residue at position 32 (which is amino acid residue 11 for SEQ ID NOs 12, 13, 14 and 15) is Val, Thr or Glu; the amino acid residue at position 33 (which is amino acid residue 12 for SEQ ID NOs 12, 13, 14 and 15) Phe or Arg; the amino acid residue at position 34 (which is amino acid residue 13 for SEQ ID NOs 12, 13, 14 and 15) is Met or Leu; the amino acid residue at position 36 (which is amino acid residue 15 for SEQ ID NOs 12, 13, 14 and 15) is Thr or Ser; the amino acid residue at position 40 (which is amino acid residue 19 for SEQ ID NOs 12, 13, 14 and 15) is Ile or Val.

4. An expression vector comprising the following operably linked elements:
a transcription promoter;
a DNA segment encoding a mammalian polypeptide, said polypeptide being comprised of SEQ ID NO: 2, 5, 11, 12, 13, 14 or 15 or a polypeptide which is at least 90% identical to said polypeptides; and
a transcription terminator.

5. An isolated polypeptide comprised of a mammalian polypeptide, said polypeptide being comprised of SEQ ID NO: 2, 5, 11, 12, 13, 14 or 15.

6. An isolated polypeptide of claim 5 wherein the polypeptide is at least 90% identical to said polypeptides of claim 5.

7. An isolated polypeptide of claim 5 having the following amino acid residue variability in SEQ ID NO:2 and 11: the amino acid residue at position 31 (which is amino acid residue 10 for SEQ ID NOs 12, 13, 14 and 15) is Trp or Thr; the amino acid residue at position 32 (which is amino acid residue 11 for SEQ ID NOs 12, 13, 14 and 15) is Val, Thr or Glu; the amino acid residue at position 33 (which is amino acid residue 12 for SEQ ID NOs 12, 13, 14 and 15) Phe or Arg; the amino acid residue at position 34 (which is amino acid residue 13 for SEQ ID NOs 12, 13, 14 and 15) is Met or Leu; the amino acid residue at position 36 (which is amino acid residue 15 for SEQ ID NOs 12, 13, 14 and 15) is Thr or Ser; the amino acid residue at position 40 (which is amino acid residue 19 for SEQ ID NOs 12, 13, 14 and 15) is Ile or Val.

8. An antibody that specifically binds to a mammalian polypeptide, said polypeptide being SEQ ID NO: 2, 5, 11, 12, 13, 14 or 15 or a polypeptide which is at least 90% identical to said polypeptides.

9. An anti-idiotypic antibody of an antibody which specifically binds to a mammalian polypeptide said polypeptide being comprised of SEQ ID NO: 2, 5, 11, 12, 13, 14 or 15 or a polypeptide which is at least 90% identical to said polypeptides.

10. A cell or bacterium which is transformed or transfected with an expression vector containing a polynucleotide which encodes a mammalian polypeptide said polypeptide being comprised of SEQ ID NO: 2, 5, 11, 12, 13, 14 or 15 or a polypeptide which is at least 90% identical to said polypeptides.


SEQUENCE LISTING (15 sequences)

SEQ ID NO: 1 - DNA, 371 bp, Homo sapiens
  CDS (126)...(371)
gaattcggctcgagcttcctcggctggatttaaggttgccgctagccgcctgggaatttaagggacccacactaccttcccgaagttgaaggcaagcggtgattgtttgtagacggcgctttgtcatgggacctgtgcggttgggaatattgcttttcctttttttggccgtgcacgaggcttgggctgggatgttgaaggaggaggacgatgacacagaacgcttgcccagcaaatgcgaagtgtgtaagctgctgagcacagagctacaggcggaactgagtcgcaccggtcgatctcgagaggtgctggagctggggcaggtgctggatacaggcaagaggaagagacacgtgccttacagcgtttcg

SEQ ID NO: 2 - protein, 82 aa, Homo sapiens
MGPVRLGILLFLFLAVHEAWAGMLKEEDDDTERLPSKCEVCKLLSTELQAELSRTGRSREVLELGQVLDTGKRKRHVPYSVS

SEQ ID NO: 3 - DNA, 443 bp, Homo sapiens
  misc_feature (1)...(443): n is any nucleotide
cgctancgcctgggaatttaagggacccacactaccttcccgaagttgaaggcaagcggtgattgtyygtagacggcgctttgtcatgggacctgtgcggttgggaatattgcttttcctttttttggccgtgcacgaggcttgggctgggatgttgaaggaggaggacgatgacacagaacgcttgcccagcaaatgcgaagtgtgtaagctgctgagcacagagctacaggcggaactgagtcgcaccggtcgatctcgagaggtgctggagctgggrcaggtgctggatacaggcaagaggaagagacacgtgccttacagcgtttcagagacaaggctggaagaggccttagagaatttatgtgagcggatcctggactatagtgttcacgctgagcgcaagggctcactganatatgccaanggtcagaatcaaacca

SEQ ID NO: 4 - DNA, 1623 bp, Homo sapiens
gaattcggctcgagcttcctcggctggatttaaggttgccgctagccgcctgggaatttaagggacccacactaccttcccgaagttgaaggcaagcggtgattgtttgtagacggcgctttgtcatgggacctgtgcggttgggaatattgcttttcctttttttggccgtgcacgaggcttgggctgggatgttgaaggaggaggacgatgacacagaacgcttgcccagcaaatgcgaagtgtgtaagctgctgagcacagagctacaggcggaactgagtcgcaccggtcgatctcgagaggtgctggagctggggcaggtgctggatacaggcaagaggaagagacacgtgccttacagcgtttcgtgagtccttcgtgctcctcccctttccaacccccaacggagccctgggagttcctacagcatccagggaatctttgtttcctctctttccacagagagacaaggctggaagaggccttagagaatttatgtgagcggatcctggactatagtgttcacgctgagcgcaagggctcactgagatatgccaagggtcagagtcagaccatggcaacactgaaaggcctagtgcagaagggggtgaaggtggatctggggatccctctggagctttgggatgagcccagcgtggaggtcacatacctcaagaagcagtgtgagaccatgttggaggagtttgaagacattgtgggagactggtacttccaccatcaggagcagcccctacaaaattttctctgtgaaggtcatgtgctcccagctgctgaaactgcatgtctacaggaaacttggactggaaaggagatcacagatggggaagagaaaacagaaggggaggaagagcaggaggaggaggaggaagaggaggaagaggaagggggagacaagatgaccaagacaggaagccaccccaaacttgaccgagaagatctttgacccttgcctttgagcccccaggaggggaagggatcatggagagccctctaaagcctgcactctccctgctccacagctttcagggtgtgtttatgagtgactccacccaagcttgtagctgttctctcccatctaacctcaggcaagatcctggtgaaacagcatgacatggcttctggggtggagggtgggggtggaggtcctgctcctagagatgaactctatccagccccttaattggcaggtgtatgtgctgacagtactgaaagctttcctctttaactgatcccacccccacccaaaagtcagcagtggcactggagctgtgggctttggggaagtcacttagctccttaaggtctgtttttagacccttccaaggaagaggccagaacggacattctctgcgatctatatacattgcctgtatccaggaggctacacaccagcaaaccgtgaaggagaatgggacactgggtcatggcctggagttgctgataatttaggtgggatagatacttggtctacttaagctcaatgtaacccagagcccaccatatagttttataggtgctcaattttctatatcgctattaaacttttttctttttttctaaaaaaaaaaaaaaaaaaaaaaaaaaaaagggcggccgccg

SEQ ID NO: 5 - protein, 33 aa, Homo sapiens
CEVCKLLSTELQAELSRTGRSREVLELGQVLDT

SEQ ID NO: 6 - DNA, 21 bp, Homo sapiens
acactatagtccaggatccgc

SEQ ID NO: 7 - DNA, 21 bp, Homo sapiens
gacccacactaccttcccgaa

SEQ ID NO: 8 - DNA, 18 bp, Homo sapiens
agcggtgattgtttgtag

SEQ ID NO: 9 - DNA, 18 bp, Homo sapiens
tgggcaagcgttctgtgt

SEQ ID NO: 10 - DNA, 1457 bp, Homo sapiens
  CDS (63)...(806)
gacccacactaccttcccgaagttgaaggcaagcggtgattgtttgtagacggcgctttgtcatgggacctgtgcggttgggaatattgcttttcctttttttggccgtgcacgaggcttgggctgggatgttgaaggaggaggacgatgacacagaacgcttgcccagcaaatgcgaagtgtgtaagctgctgagcacagagctacaggcggaactgagtcgcaccggtcgatctcgagaggtgctggagctggggcaggtgctggatacaggcaagaggaagagacacgtgccttacagcgtttcagagacaaggctggaagaggccttagagaatttatgtgagcggatcctggactatagtgttcacgctgagcgcaagggctcactgagatatgccaagggtcagagtcagaccatggcaacactgaaaggcctagtgcagaagggggtgaaggtggatctggggatccctctggagctttgggatgagcccagcgtggaggtcacatacctcaagaagcagtgtgagaccatgttggaggagtttgaagacattgtgggagactggtacttccaccatcaggagcagcccctacaaaattttctctgtgaaggtcatgtgctcccagctgctgaaactgcatgtctacaggaaacttggactggaaaggagatcacagatggggaagagaaaacagaaggggaggaagagcaggaggaggaggaggaagaggaggaagaggaagggggagacaagatgaccaagacaggaagccaccccaaacttgaccgagaagatctttgacccttgcctttgagcccccaggaggggaagggatcatggagagccctctaaagcctgcactctccctgctccacagctttcagggtgtgtttatgagtgactccacccaagcttgtagctgttctctcccatctaacctcaggcaagatcctggtgaaacagcatgacatggcttctggggtggagggtgggggtggaggtcctgctcctagagatgaactctatccagccccttaattggcaggtgtatgtgctgacagtactgaaagctttcctctttaactgatcccacccccacccaaaagtcagcagtggcactggagctgtgggctttggggaagtcacttagctccttaaggtctgtttttagacccttccaaggaagaggccagaacggacattctctgcgatctatatacattgcctgtatccaggaggctacacaccagcaaaccgtgaaggagaatgggacactgggtcatggcctggagttgctgataatttaggtgggatagatacttggtctacttaagctcaatgtaacccagagcccaccatatagttttataggtgctcaattttctatatcgctattaaacacctgcccgggcggccgctcgagccctatagtgagtcgtattaggatgg

SEQ ID NO: 11 - protein, 248 aa, Homo sapiens
MGPVRLGILLFLFLAVHEAWAGMLKEEDDDTERLPSKCEVCKLLSTELQAELSRTGRSREVLELGQVLDTGKRKRHVPYSVSETRLEEALENLCERILDYSVHAERKGSLRYAKGQSQTMATLKGLVQKGVKVDLGIPLELWDEPSVEVTYLKKQCETMLEEFEDIVGDWYFHHQEQPLQNFLCEGHVLPAAETACLQETWTGKEITDGEEKTEGEEEQEEEEEEEEEEGGDKMTKTGSHPKLDREDL

SEQ ID NO: 12 - protein, 211 aa, Homo sapiens
CEVCKLLSTELQAELSRTGRSREVLELGQVLDTGKRKRHVPYSVSETRLEEALENLCERILDYSVHAERKGSLRYAKGQSQTMATLKGLVQKGVKVDLGIPLELWDEPSVEVTYLKKQCETMLEEFEDIVGDWYFHHQEQPLQNFLCEGHVLPAAETACLQETWTGKEITDGEEKTEGEEEQEEEEEEEEEEGGDKMTKTGSHPKLDREDL

SEQ ID NO: 13 - protein, 61 aa, Homo sapiens
GMLKEEDDDTERLPSKCEVCKLLSTELQAELSRTGRSREVLELGQVLDTGKRKRHVPYSVS

SEQ ID NO: 14 - protein, 49 aa, Homo sapiens
GMLKEEDDDTERLPSKCEVCKLLSTELQAELSRTGRSREVLELGQVLDT

SEQ ID NO: 15 - protein, 227 aa, Homo sapiens
GMLKEEDDDTERLPSKCEVCKLLSTELQAELSRTGRSREVLELGQVLDTGKRKRHVPYSVSETRLEEALENLCERILDYSVHAERKGSLRYAKGQSQTMATLKGLVQKGVKVDLGIPLELWDEPSVEVTYLKKQCETMLEEFEDIVGDWYFHHQEQPLQNFLCEGHVLPAAETACLQETWTGKEITDGEEKTEGEEEQEEEEEEEEEEGGDKMTKTGSHPKLDREDL